(19) United States
(12) Patent Application Publication
(10) Pub. No.: US 2009/0324757 A1
(43) Pub. Date: Dec. 31, 2009

(54) Compositions and Methods for Modulating Visceral Sensation and/or Gastrointestinal Reflex Activity

(75) Inventors: Svyatoslav Komarnytsky, Highland Park, NJ (US); Dmitry Govorko, Princeton, NJ (US); Debora A. Esposito, Somerset, NJ (US); Ilya Raskin, Manalapan, NJ (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: RUTGERS, THE STATE UNIVERSITY OF NEW JERSEY, New Brunswick, NJ (US)

(21) Appl. No.: 12/415,430

(22) Filed: Mar. 31, 2009

Related U.S. Application Data
(60) Provisional application No. 61/041,118, filed on Mar. 31, 2008.

(51) Int. Cl. A61K 36/27; A61K 31/56; A61P 1/00

(52) U.S. Cl. 424/773; 424/725; 514/169; 424/774; 424/779; 424/778; 424/777

(57) ABSTRACT

The invention relates to compositions capable of modulating visceral sensation and/or gastrointestinal activity obtained from botanical sources. More specifically, the invention relates to compositions comprising an extract product of an Asclepias plant, and methods of modulating visceral sensation and/or gastrointestinal activity in an individual using same.

### CROSS REFERENCE TO RELATED APPLICATION

The benefit under 35 U.S.C. § 119(e) of U.S. provisional patent application Ser. No. 61/041,118, filed Mar. 31, 2008, the entire disclosure of which is incorporated herein by reference, is hereby claimed.

### FIELD OF THE INVENTION

The invention relates to compositions obtained from botanical sources. More specifically, the invention relates to compositions comprising an extract product of an Asclepias plant, and methods for modulating visceral sensation and/or gastrointestinal activity by administering said compositions.

### BACKGROUND OF THE INVENTION

Pregnanes are a class of naturally occurring saturated steroid substances which are widely distributed in the plant kingdom. Pregnanes exhibit a wide range of biological activity including antitumor, cytotoxic, platelet pro-aggregating, antifungal, acetylcholine esterase inhibiting, and anti-osteoporotic properties [Dal Piaz, Rapid Comm. Mass Spec. 19(8):1041-52 (2005)].

Certain botanical compositions comprising pregnane glycosides obtained from Hoodia, Stapelia, Orbea, and Asclepias plants have been shown to possess appetite-suppressing activity [U.S. Pat. No. 6,376,657 to Van Heerden et al., U.S. Pat. No. 7,008,648 to Corley et al., and U.S. patent No. to Raskin et al.]. The site and mechanism of action for these compositions have not been demonstrated and are not well understood.

Serotonin (5-hydroxytryptamine or 5-HT) has complex actions within the gastrointestinal system. 5-HT acts as a transducer of signals from the gut lumen to the terminals of afferent nerve fibers that lie in close proximity thereto, including the vagus nerve [Andrews et al., Curr. Opin. Pharmacol., 2(6):650-656 (2002)]. 5-HT acts through a large family of seven classes of receptors that includes ligand-gated ion channels and G protein-coupled receptors [Hoyer et al., Pharmacol. Biochem. Behav., 71(4): 533-54 (2002)]. 5-HT type 3 receptors belong to the former group and are implicated in the transmission of extrinsic afferent signals, particularly those following a vagal pathway [Kirkup et al., Am. J. Physiol. Gastrointest. Liver Physiol., 280(5): G787-94 (2001)].

5-HT type 3 receptor antagonists and 5-HT type 4 receptor agonists have been used to treat irritable bowel syndrome [Tonini et al., Dig Dis., 24(1-2):59-69 (2006)]. Additionally, 5-HT type 3 receptor agonists and partial agonists have been shown to modify gastrointestinal motility in rodents, dogs, and humans [Chetty et al., Br. J. Pharmacol., 148(7):1012-21 (2006); Nagakura et al., Eur. J. Pharmacol., 327(2-3):189-93 (1997); and Coleman et al., Aliment. Pharmacol. Ther., 18(10):1039-48 (2003)]. For example, Coleman et al. demonstrated that administration of a selective 5-HT type 3 receptor agonist delayed gastric emptying and accelerated small intestinal transit in humans.

Accordingly, compositions capable of modulating 5-HT receptor activity are of considerable interest.

### SUMMARY OF THE INVENTION

The invention provides methods for modulating visceral sensation and gastrointestinal activity. The disclosed methods can be used to treat individuals who suffer from various gastrointestinal disorders and to safely provide relief therefrom.

One embodiment according to the invention provides methods of treating a gastrointestinal condition comprising administering a therapeutically effective amount of a composition containing an extract product of a plant material of an Asclepias plant to an individual having a gastrointestinal condition.

In a further embodiment, the invention provides methods of inhibiting gastric acid secretion comprising administering a therapeutically effective amount of a composition containing an extract product of a plant material of an Asclepias plant to an individual in need of gastric acid secretion inhibition.

In yet a further embodiment, the invention provides methods of treating an evacuation disorder comprising administering a therapeutically effective amount of a composition containing an extract product of a plant material of an Asclepias plant to an individual having an evacuation disorder.

### DETAILED DESCRIPTION OF THE INVENTION

The invention demonstrates that compositions containing an extract product of an Asclepias plant are capable of modulating visceral sensation and gastrointestinal reflex activity. Any composition containing an extract product in accordance with the invention, for example, solutions or dried preparations, may be used as a natural alternative to treat various gastrointestinal conditions. Accordingly, the disclosed compositions can be administered to effectively treat various gastrointestinal conditions.

The invention also provides methods of administering compositions comprising pregnane derivatives according to formulae I, II, III, IV, and/or V (formulae set forth below) which exhibit therapeutic and physiological effects by modulating at least one class of 5-hydroxytryptamine (5-HT) receptors, for example, at least one of 5-HT type 1 receptors, 5-HT type 2 receptors, 5-HT type 3 receptors, 5-HT type 4 receptors, 5-HT type 5 receptors, 5-HT type 6 receptors, and 5-HT type 7 receptors.

The invention further provides methods of administering compositions comprising pregnane derivatives according to formulae I, II, III, IV, and/or V (formulae set forth below) to increase visceral afferent signaling, for example, via the vagus cholinergic pathway. It is well known that modulating the vagus nerve is highly beneficial in many pathological conditions, for example, by inhibiting pro-inflammatory cytokine release. Accordingly, the compositions of the invention can be administered to protect against systemic inflammation, or to treat individuals having anti-inflammatory disorders. Similarly, modulating the vagus nerve can be used to achieve cardioinhibition without adversely affecting heart rate, respiration, and/or hemodynamics. Accordingly, the compositions of the invention can be administered to treat individuals in need of cardioinhibition (for example, individuals having atrial fibrillation with rapid ventricular response or tachycardia). Further, modulating the vagus nerve can be used to treat mood disorders, depression, anxiety, migraines, and Alzheimer's disease. Accordingly, the compositions of the invention can be administered to treat individuals having mood disorders, depression, anxiety, migraines, and Alzheimer's disease. Finally, modulating the vagus nerve can influence thermoregulation. Accordingly, the compositions of the invention can be administered to individuals in order to either depress or stimulate metabolic processes.

As used herein, the term “extract product” refers to any compound, any agent, and/or mixtures thereof, that is obtained, isolated, and/or derived from an extract of a plant material. The term “plant material” refers to any plant material including, but not limited to, leaves, stems, flowers, fruits, seeds, roots, and combinations thereof. In one embodiment, the plant material includes roots only. In another embodiment, the plant material includes the above-ground portions of the plant only.

As used herein, the term “gastrointestinal condition” refers to structural (or mucosal) gastrointestinal tract disorders and non-structural (or non-mucosal) gastrointestinal tract disorders.

The terms “structural gastrointestinal tract disorder” or “mucosal gastrointestinal tract disorder” refer to any gastrointestinal tract disorder related to structural or mucosal abnormalities of the gastrointestinal tract or any gastrointestinal tract disorder where there is evidence of a related metabolic disturbance, including but not limited to inflammatory bowel disorders, structural gastroesophageal disorders, and structural intestinal disorders.

The terms “non-structural gastrointestinal tract disorder” or “non-mucosal gastrointestinal tract disorder” refer to any gastrointestinal tract disorder not related to structural or mucosal abnormalities of the gastrointestinal tract, or any gastrointestinal tract disorder where there is no evidence of a related metabolic disturbance, including but not limited to functional gastrointestinal tract disorders. Accordingly, the term “functional gastrointestinal tract disorder” refers to any gastrointestinal tract disorder associated with a disturbance of motor or sensory function in the absence of mucosal or structural damage or any gastrointestinal tract disorder associated with a disturbance of motor or sensory function in the absence of a metabolic disorder. Functional gastrointestinal tract disorders include functional dysphagia, non-ulcer dyspepsia, irritable bowel syndrome (IBS), slow-transit constipation, and evacuation disorders.

“Evacuation disorder” as used herein refers to any disorder where defecation occurs poorly, e.g., an individual is unable to expel stool. The term “slow-transit constipation” refers to an exemplary evacuation disorder involving slowing of motility in the large intestine with a prolonged transit time through the organ.

Gastrointestinal conditions treatable with compositions according to the invention typically involve delayed small intestinal and/or colonic transit, excess gastric acid production, and/or rapid gastric emptying. Representative conditions include but are not limited to ileus, intestinal pseudoobstruction, Ogilivie syndrome, Hirschsprung's disease, intestinal neuronal dysplasia, gastroparesis, acid reflux disease, irritable bowel syndrome, inflammatory bowel disease, dyspepsia, emesis, and constipation.

The term “therapeutically effective amount” refers to an amount of a composition containing an Asclepias extract product that is sufficient to modulate visceral sensation and/or gastrointestinal activity in an individual. Alternatively, the term “therapeutically effective amount” refers to an amount of composition containing an Asclepias extract product that is sufficient to alleviate, ameliorate, prevent, and/or clear the symptoms and/or the pathology of a gastrointestinal condition.

“Modulate” refers to a statistically significant and detectable or measurable inhibiting, activating, modifying, regulating, or controlling influence on a receptor demonstrated by administering a composition or compound according to the invention. For example, modulating a 5-HT receptor refers to a statistically significant and detectable or measurable change in 5-HT receptor activity in an individual (over a time period of at least about 1 hour).

“Visceral afferent signaling” is intended herein to encompass activation of vagal and spinal afferents that convey sensory information from the gut to the central nervous system and are involved in control of gastrointestinal activities, such as pancreatic secretion, gut motility, gastrointestinal blood flow, detection of intestinal chemicals, endocrine signals, and monitoring of gastric distension.

Typically, the compositions according to the invention contain an extract product of a plant of the genus Asclepias. Advantageously, Asclepias plants are easy to cultivate and propagate, and provide excellent sources of plant material. As used herein, the term “easily cultivatable” refers to a plant which is relatively easy to germinate, reasonably resistant to pathogens, responds well to conventional agronomic practices, produces significant amounts of plant material, and is capable of producing multiple crops in a single growing season in the field (or in a year in greenhouse conditions). Plants having such qualities include conventionally known and commercially available vegetable crops. In one aspect according to the invention, easily cultivatable plants are plants which grow from seed to maturity in less than about 1 year, preferably less than about one-half year, and more preferably in about one-quarter year. In another aspect, easily cultivatable plants are plants which are capable of providing greater than about 0.5 grams, preferably more than about 1.0 grams, more preferably more than about 1.5 grams, and even more preferably more than about 2.0 grams of fresh plant material in one month per plant. In yet another aspect, easily cultivatable plants are plants which occupy less than about 25 cm², preferably less than about 15 cm², and more preferably less than about 10 cm² of area per plant. In an additional aspect, easily cultivatable plants are plants which are capable of providing greater than about 500 grams, preferably more than about 1.0 kilogram, more preferably more than about 1.5 kilograms, and even more preferably more than about 2.0 kilograms of fresh plant material per square meter in one year.

Asclepias plants in accordance with the invention include, but are not limited to, A. incarnata, A. curassayica, A. syriaca, and A. tuberosa. Typically, the plants are selected from the group consisting of A. incarnata, A. curassayica, and A. syriaca.

As demonstrated herein, Asclepias plants produce compounds and/or agents capable of modulating visceral sensation and gastrointestinal activity. The visceral sensation and/or gastrointestinal activity of Asclepias plants (and thus of the disclosed compositions) is generally attributed to the presence of one or more compounds in accordance with the following formulae I, II, and III:

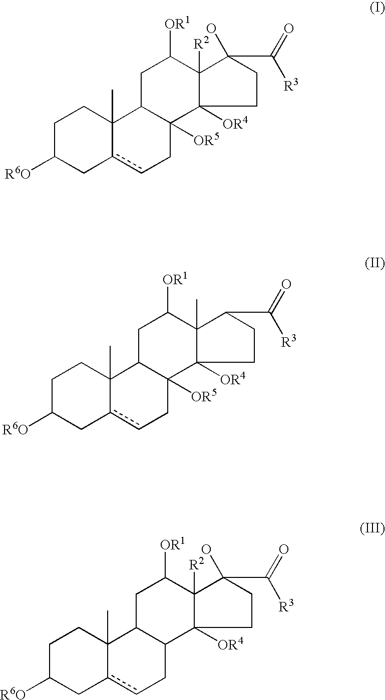

wherein R¹ is hydrogen or a C₁-C₁₈ moiety;

R² (if present) is hydrogen or a C₁-C₁₈ moiety;

R³ is a C₁-C₁₈ moiety;

R⁴ is hydrogen or a C₁-C₁₈ moiety;

R⁵ (if present) is hydrogen or a C₁-C₁₈ moiety;

R⁶ is hydrogen, a C₁-C₁₈ moiety, or a saccharide moiety; and,

the dotted line represents an optional double bond.

As used herein, the term “C₁-C₁₈ moiety” includes from one to 18 carbon atoms. Typical examples include alkyl, alkylene, heteroalkyl, alkenyl, acyl, and aryl groups as defined herein. Of course, in some embodiments, an individual substituent may be described by more than one of the aforementioned terms.

“Alkyl” as used herein includes straight chain and branched hydrocarbon groups containing up to 18 carbon atoms, for example, one to ten, and one to eight carbon atoms.

“Alkylene” as used herein refers to alkyl groups (as defined) further including one or more substituents.

Additionally, “heteroalkyl” as used herein refers to alkyl groups further containing a heteroatom such as 0, P, S, or N.

“Alkenyl” as used herein refers to alkyl groups further containing one or more carbon-carbon double bonds.

“Acyl” as used herein refers to a substituent having the chemical formula A:

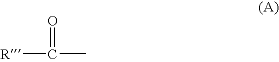

wherein R′″ is a moiety, as defined above, but containing between one and 17 carbon atoms. In preferred embodiments, R′″ is selected from the group consisting of aryl and alkylene, particularly alkylenearyl (i.e., an alkylene group having an aryl substituent). Representative acyl groups include formyl, acetyl, propionyl, butyryl, benzoyl, toluoyl, phenylacetyl, tigloyl, and cinnamoyl. In some embodiments, R¹ is preferably an acyl group, particularly benzoyl, tigloyl, or cinnamoyl. In some embodiments, the acyl group includes an aryl group containing a heteroatom, as described below.

The term “aryl” is defined herein as a monocyclic or polycyclic aromatic group, preferably a monocyclic or bicyclic aromatic group, e.g., phenyl or naphthyl. An “aryl” group can be unsubstituted or substituted, for example, with one or more, and in particular one to three, halo, alkyl, alkoxy, alkoxyalkyl, haloalkyl, nitro, and/or cyano substituents. The aryl group may also contain one or more heteroatoms such as O, P, S, or N.

The term “saccharide moiety” as used herein refers to a pentose, hexose, heptose, or octose sugar, analog, or derivative thereof, including, but not limited to, deoxy sugars, dideoxy sugars, amino sugars, and sugar acids. The term includes disaccharides, oligosaccharides, and polysaccharides, which are comprised of two or more saccharides that are joined by a glycosidic linkage.

Compounds capable of modulating visceral sensation and/or gastrointestinal activity in accordance with formulae I, II, and III include compounds in accordance with the following formula IV:

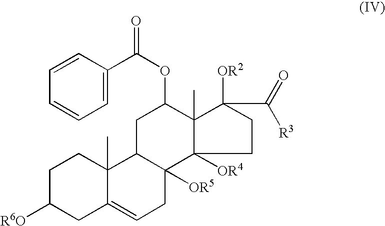

wherein R² is hydrogen or a C₁-C₁₈ moiety;

R³ is a C₁-C₁₈ moiety;

R⁴ is hydrogen or a C₁-C₁₈ moiety;

R⁵ is hydrogen or a C₁-C₁₈ moiety; and,

R⁶ is hydrogen, a C₁-C₁₈ moiety, or a saccharide moiety.

Compounds capable of modulating visceral sensation and/or gastrointestinal activity in accordance with formulae I, II, and III also include compounds in accordance with the following formula V:

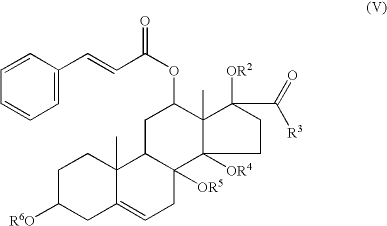

wherein R² is hydrogen or C₁-C₁₈ moiety;

R³ is C₁-C₁₈ moiety;

R⁴ is hydrogen or a C₁-C₁₈ moiety;

R⁵ is hydrogen or a C₁-C₁₈ moiety; and,

R⁶ is hydrogen, a C₁-C₁₈ moiety, or a saccharide moiety.

Compounds in accordance with formulae I, II, III, IV, and/or V are steroidal compounds. In one embodiment, the steroidal compounds are pregnane glycosides (i.e., R⁶ is a saccharide moiety). In another embodiment, the steroidal compounds are pregnane aglycones (i.e., R⁶ is hydrogen).

In another embodiment, isolated and purified compounds in accordance with formulae I, II, III, IV, and/or V, or pharmaceutically acceptable salts, solvates, or hydrates thereof, could be used to treat gastrointestinal conditions, e.g., by administering a therapeutically effective amount of one or more such compounds to an individual having a gastrointestinal condition. Similarly, one or more isolated and purified compounds of formulae I, II, III, IV, and/or V, or pharmaceutically acceptable salts, solvates, or hydrates thereof, could be used to treat individuals in need of gastric acid secretion inhibition, and/or individuals having an evacuation disorder.

The disclosed compositions capable of modulating visceral sensation and/or gastrointestinal activity typically contain a mixture of compounds in accordance with formulae I, II, III, IV, and/or V. Accordingly, the invention contemplates mixtures, which may exhibit additive, or preferably synergistic, effects.

Asclepias plants are grown and harvested using well-known methods. For example, the plants may be grown in an agricultural field. More preferably, the plants are grown in environmentally controlled hydroponic greenhouses using standard hydroponic methods. Hydroponic methods facilitate the reproducible optimization of plant growing conditions, and therefore the content of compounds capable of modulating visceral sensation and gastrointestinal activity. Hydroponic methods also facilitate harvesting of the plants. Additionally, controlled growth conditions are advantageous in that they facilitate the standardization of any final product.

The conditions under which the plants are grown may also affect the content of compounds capable of modulating visceral sensation and gastrointestinal activity. In particular, plants subjected to stress conditions, such as heat stress, dehydration, physical wounding, and/or exposed to chemical elicitors, are expected to have a higher content of compounds capable of modulating visceral sensation and/or gastrointestinal activity than plants not subjected to such conditions. Any conventionally known chemical elicitor can be used during cultivation of the Asclepias plants, in accordance with known application schedules.

As previously described, the compounds capable of modulating visceral sensation and/or gastrointestinal activity are typically isolated by extracting plant material of an Asclepias plant. Any plant material, including leaves, stems, flowers, fruits, roots, and combinations thereof, can be extracted. In one embodiment, the above-ground plant parts are extracted. In another embodiment, the plant roots are used.

One exemplary extraction method for obtaining high yields of compounds capable of modulating visceral sensation and/or gastrointestinal activity from Asclepias plants in accordance with the invention comprises the following steps: (1) providing fresh or fresh-frozen plant material; (2) disrupting the plant material; and (3) extracting the plant material in a solution containing a sufficient amount of solvent; and (4) isolating the extract. The extract may be further processed by: (5) removing solid matter from the extract; (6) removing solvent components; (7) resuspending the resulting residue in an aqueous solution; and (8) after removing any water insoluble material, repeating step (6) to form a more purified form of an extract product. In various embodiments, the plant material can be disrupted by macerating, grinding, or otherwise disrupting the plant material.

In a preferred embodiment, fresh plant tissue is quick-frozen in liquid nitrogen, then ground or otherwise macerated (e.g., using a Polytron or a Waring blender) in solvent. After solids are removed from the extract, e.g., by filtration, centrifugation, or any method known in the art, the content of compounds capable of modulating visceral sensation and/or gastrointestinal activity (of the extract) can optionally be measured by any known method, including spectrometric methods.

Solvents for use in the extraction methods of the invention include well-known organic solvents such as, but not limited to, water, alcohols, alkanes, halocarbons, ethers, aromatic solvents, ketones, aqueous solvents, esters, and super critical fluids. In one embodiment, ethanol is a preferred alcohol for practice of the invention. A benefit of incorporating an ethanolic solvent in the final extraction step is that an ethanolic solvent is compatible with an ingestible product, and therefore is suitable for incorporation into a pill, capsule, tablet, and other ingestible forms known in the art.

A composition containing an Asclepias extract product can be tableted, encapsulated or otherwise formulated for oral administration (e.g., in a gum or candy). The compositions may be provided as pharmaceutical compositions (e.g., an ethical drug), nutraceutical compositions (e.g., a dietary supplement), or as a food or beverage additive, as defined by the U.S. Food and Drug Administration. The dosage form for the above composition is not particularly restricted; for example, liquid solutions, suspensions, emulsions, tablets, pills, capsules, sustained release formulations, powders, suppositories, liposomes, microparticles, microcapsules, sterile isotonic aqueous buffer solutions, etc. can be used.

The compositions typically include one or more suitable diluents, fillers, salts, disintegrants, binders, lubricants, glidants, wetting agents, controlled release matrices, colorants, flavoring, carriers, excipients, buffers, stabilizers, solubilizers, commercial adjuvants, and/or other additives known in the art.

Any pharmaceutically acceptable (i.e., sterile and acceptably non-toxic as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Such compositions may influence the physical state, stability, rate of in vivo release, and rate of in vivo clearance of compounds capable of modulating visceral sensation and gastrointestinal activity.

Pharmaceutically acceptable fillers can include, for example, lactose, microcrystalline cellulose, dicalcium phosphate, tricalcium phosphate, calcium sulfate, dextrose, mannitol, and/or sucrose. Salts, including calcium triphosphate, magnesium carbonate, and sodium chloride, may also be used as fillers in the pharmaceutical compositions.

Binders may be used to hold the composition containing the extract product together to form a hard tablet. Exemplary binders include materials from natural products such as acacia, tragacanth, starch and gelatin. Other suitable binders include methyl cellulose (MC), ethyl cellulose (EC) and carboxymethyl cellulose (CMC).

The methods in accordance with the invention contemplate administration of an Asclepias extract product containing composition whether or not symptoms are manifest, i.e., prophylactic administration is contemplated. The compositions of the invention can be used alone or in conjunction with other therapies including, for example, administration of other therapeutic agents capable of modulating visceral sensation and gastrointestinal activity.

It will be appreciated that the treatment methods of the invention are useful in the fields of human medicine and veterinary medicine. Thus, the subject or individual to be treated may be a mammal, preferably human, or other animals. For veterinary purposes, subjects include, for example, farm animals such as cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

In the methods according to the invention, the compositions capable of modulating visceral sensation and/or gastrointestinal activity may be administered by any known route of administration. For example, a composition containing an extract product of an Asclepias plant can be formulated for injection, or for oral, nasal, transdermal, intramuscular, intraperitoneal, intravenous, subcutaneous, inhalation, intralesional, endothelial, topical, by oral mucosa, rectally, by intestinal mucosa, or other forms of administration. Typically, the compositions are formulated for topical, oral, or nasal administration, particularly for oral administration. In some embodiments, the compositions are prepared using a non-toxic alcohol or an aqueous solution.

Because preferred dosages of therapeutics for modulating visceral sensation and/or gastrointestinal activity such as Tegaserod and Alosetron are known in the art for a variety of therapeutic and prophylactic purposes, appropriate dosages of the compositions in accordance with the invention may be easily determined by standard methods. The amount and dosage regimen of the compositions according to the invention are based on various factors relevant to the purpose of administration, for example human or animal age, sex, body weight, hormone levels, or other needs for a particular treatment or prophylaxis. A typical dosage is about 0.01 mg/kg body weight/day to about 200 mg/kg body weight/day, preferably about 5 mg kg body weight/day to about 50 mg/kg body weight/day. A typical treatment course may comprise multiple doses of a composition containing an amount of an Asclepias extract product effective to modulate visceral sensation and/or gastrointestinal activity in an individual on a daily basis for significant periods of time, for example, three doses per day for three months. In one embodiment, a presently preferred dose amount is one dose per day. The compositions may be administered to an individual at any time. Typically, the compositions are administered concurrently or prior to consumption of a meal. Of course, these are only exemplary treatment schedules, and other schedules are contemplated. In each case, the suitability of such schedules and the aforementioned modes of administration are determined by those of skill in the art, using routine procedures. For example, those of skill in the art will be able to take the information disclosed in this specification and optimize treatment regimes for human subjects based on clinical trials performed in accordance with the specification.

The following Examples are provided to describe the invention in greater detail, and are intended to illustrate, not to limit, the appended claims.

### Example 1

### Methods of Extracting an Asclepias Plant Material

Two exemplary methods for extracting an Asclepias plant material, thereby providing a composition containing an extract product and having compounds capable of modulating visceral sensation and gastrointestinal activity, are described. The first extraction method provides extract products rich in pregnane glycosides. The second extraction method provides extract products rich in pregnane aglycones, possibly because of the hydrolysis of the saccharide moieties represented, for example, by R⁶ in formulae I, II, III, IV, and V above. Additionally, extracts obtained using both protocols were analyzed by liquid chromatography mass spectroscopy (“LC-MS”) to provide additional information regarding the compositions containing extract products.

In the first method, fresh hydroponically grown root material was frozen at −20° C. The frozen root material was lyophilized for 48 hours until dry. The dried root material was ground by mortar and pestle. Ten grams of root material were then extracted for 24 hours in 0.5 liters of a mixture of methylene chloride and methanol (about 1 part methylene chloride to about 1 part methanol by volume). The solid plant material was removed by filtration through Miracloth™ filter paper and the remaining solvent was removed under reduced pressure using a rotary evaporation device having a 38° C. water bath. The dried, crude extract was dissolved in 250 milliliters (ml) of ultrapure water and partitioned against 0.5 liters of methylene chloride. The methylene chloride fraction was saved and the solvent removed by rotary evaporation. The water fraction was discarded. The dry extract was dissolved in 150 ml of methanol and partitioned twice against 300 ml of hexanes. The solvent of the methanol fraction was removed by rotary evaporation. The resulting “active” fraction was dissolved in 300 ml of water, frozen at −20° C., and lyophilized to provide an extract product in the form of a dry powder.

In the second method, fresh hydroponically grown root material was frozen at −20° C. The frozen root material was lyophilized for 48 hours until dry. The dried root material was ground by mortar and pestle. Ten grams of dry root material was then extracted for 24 hours in 0.5 liters of 100% ethanol. The solid plant material was removed by filtration through Miracloth™ filter paper and the remaining solvent was removed from the extract using a rotary evaporation device having a 38° C. water bath. The dried extract was dissolved in 0.5 liters of 0.5 N HCl for 24 hours. The solid plant material was removed by filtration through the filter paper. The pH of the solution was adjusted to about 9.5 with 20-22% (approximately 45 ml) of ammonium hydroxide to provide a basic solution. 1.0 liter of chloroform was added to the basic solution. The chloroform fraction was isolated and the chloroform was removed using a rotary evaporation device. The dried extract product was stored at −20° C.

Approximately two weight percent of extract product is obtained per kilogram of fresh plant material (i.e., 20 grams of an extract product per kilogram of fresh Asclepias plant material). Further purification (e.g., by chromatographic methods) of the extract product could increase the efficacy of the extract product and/or reduce the dose needed to effectively modulate visceral sensation and/or gastrointestinal activity in an individual.

### Example 2

### Isolation and Identification of Pregnane Structures

Compositions containing extract products in accordance with the invention were analyzed to determine if they contained pregnane steroidal compounds. LC-MS analysis confirmed that pregnane structures in accordance with formulae I, II, and/or III are present in extract products of A. incarnata and A. curassayica prepared in accordance with the exemplary extraction methods of Example 1.

This Example further demonstrates that compositions in accordance with the invention typically contain a mixture of pregnane glycosides and aglycones. For example, HPLC fractionation was performed on an extract product of A. curassayica that was prepared in accordance with the second extraction method of Example 1. Subsequent analysis of fractions corresponding to several of the resulting peaks led to the structural identification of two pregnane backbones. MS fragmentation patterns and/or proton and carbon thirteen NMR experiments were used to identify the specific pregnane backbones.

### Example 3

### Demonstration of Delayed Gastric Emptying

Twenty four male Wistar rats, each weighing approximately 260 grams, were randomly divided into four groups of six animals each. The rats were individually caged in a temperature controlled environment (22° C.) with 12 h light/12 h dark cycle; lights off at 14:00; food (Purina Rodent Chow) and water were available on an ad libitum basis from day 0 to day 7 of the experiment.

The first group (control group C) was gavaged with 5% DMSO in 1 ml of corn oil; the second group (group E100) was gavaged with 100 mg/kg of an extract product in accordance with the invention in 5% DMSO in 1 ml of corn oil; the third group (group O10E100) was gavaged with 100 mg/kg of extract product in accordance with the invention and 10 mg/kg ondansetron (a known 5-HT3 receptor antagonist) in 5% DMSO in 1 ml of corn oil; and the fourth group (VAE100) included animals with ablated vagus afferents that were gavaged with 100 mg/kg of extract product in accordance with the invention in 5% DMSO in 1 ml of corn oil.

Chemical ablation of vagal afferents in VAE100 animals was achieved by subsequent subcutaneous administration of 25, 25, and 50 mg/kg capsaicin at daily intervals and animals were used 10 days after the last capsaicin administration.

After an overnight fast, on day 8, the animal groups received treatment as described above. Then animals were then allowed free access to food, and food intake was recorded 2 h following the treatment. At this point animals were sacrificed and stomach weight, volume, and gastric wall weight were recorded to estimate gastric emptying of solid meal. The results are presented in Table 1 and demonstrate that the botanical extract enriched with pregnane derivatives according to the invention significantly delayed gastric emptying of a solid meal. Because treatment with a HT type 3 selective inhibitor, ondansetron, attenuated the delayed gastric emptying observed with the compositions according to the invention, this example is consistent with the capability of compositions in accordance with the disclosure to modulate 5-hydroxytryptamine (5-HT) receptors. Additionally, because the ablation of vagal efferents also attenuated the delayed gastric emptying observed with the compositions according to the invention, this example is consistent with the capability of compositions in accordance with the disclosure to modulate visceral afferent signaling (and our understanding that at least HT-3 receptors present on the vagus nerve are being modulated).

TABLE 1
Delayed gastric emptying of solid meal.

| | Stomach | Stomach | Gastric wall | Food intake |
|---|---|---|---|---|
| Group | volume, ml | weight, g | weight, g | g/2 h |
| C | 4.6 ± 0.2 | 4.82 ± 0.51 | 1.64 ± 0.02 | 8.98 ± 0.52 |
| E100 | 12.2 ± 1.5** | 13.7 ± 1.54** | 1.74 ± 0.04 | 6.94 ± 0.70* |
| O10E100 | 6.7 ± 0.6* | 7.2 ± 0.83* | 1.69 ± 0.05 | 8.29 ± 0.43 |
| VAE100 | 5.9 ± 0.9* | 6.7 ± 1.13* | 1.71 ± 0.06 | 8.92 ± 0.47 |

**significant at p < 0.01;
*significant at p < 0.05

### Example 4

### Demonstration of Delayed Gastric Emptying

Animals were cared for and randomized into treatment groups as described in Example 3. After an overnight fast, on day 8, each animal was given intragastrically 1 ml of liquid meal comprising 0.05% phenol red (a marker dye) in either (i) a saline vehicle containing no therapeutic compositions (control group (C); a saline vehicle containing 100 mg/kg of an extract product in accordance with the invention (group E100); a saline vehicle containing 100 mg/kg of extract product in accordance with the invention and 10 mg/kg ondansetron (group 010E100); or a saline vehicle containing 100 mg/kg of extract product in accordance with the invention (VAE100).

Gastric emptying of the liquid meal was determined by the phenol red method. Thirty minutes after administration of the meal, the animals were sacrificed, and the gastroesophageal junction and pylorus were clamped. The stomachs were removed, opened, and placed in 14 ml of 0.1 N NaOH. The suspension was allowed to settle for 1 h at room temperature, and then 5 ml of the supernatant was added to 0.5 ml of 20% trichloroacetic acid (w/v) and centrifuged at 3000 rpm for 20 min. The supernatant was mixed with 4 ml of 0.5 N NaOH, and the amount of phenol red was determined from the absorbance at 560 nm. Phenol red recovered from animals that were sacrificed immediately after administration of the test meal was used as a 100% emptying standard. The results are presented in Table 1 and demonstrate that the botanical extract enriched with pregnane derivatives according to the invention significantly delayed gastric emptying of a liquid meal.

Because treatment with ondansetron (a 5-HT3 antagonist) attenuated the delayed gastric emptying observed with the compositions according to the invention, this example is consistent with the capability of compositions in accordance with the disclosure to modulate 5-hydroxytryptamine (5-HT) receptors. Additionally, because the ablation of vagal efferents substantially canceled the delayed gastric emptying observed with the compositions according to the invention, this example is also consistent with the capability of compositions in accordance with the disclosure to modulate visceral afferent signaling (and our understanding that at least HT-3 receptors present on the vagus nerve are being modulated).

TABLE 2
Delayed gastric emptying of liquid meal.

| | Gastric emptying, |
|---|---|
| Group | % of initial amount |
| C | 42.15 ± 4.88 |
| E100 | 93.80 ± 3.17** |
| O10E100 | 76.23 ± 6.54* |
| VAE100 | 53.72 ± 7.33 |

**significant at p < 0,01;
*significant at p < 0.05

### Example 5

### Demonstration of Enhanced Small Intestinal and Colonic Transit

Animals were cared for and randomized into the treatment groups according to the methods described in Example 3. After an overnight fast, on day 8, the animal groups received a liquid meal as described in Example 4.

At 60 min post-gavage, the entire gastrointestinal tract was removed, placed into ice cold saline, and small intestinal transit was measured by calculating the geometric center of the dye marker present in the small intestine only.

In a separate experiment, after an overnight fast, on day 8, the effect of orally administered botanical extract according to the invention on colonic transit was evaluated. The number of fecal pellets expelled by each animal was measured 1 h after the treatment.

The results of both experiments are summarized in Table 3 and demonstrate that the botanical extract enriched with pregnane derivatives according to the invention significantly increased small intestinal and colonic transit. Because treatment with ondansetron (a 5-HT3 antagonist) attenuated the increase in intestinal and colonic transit observed with the compositions according to the invention, this example is consistent with the capability of compositions in accordance with the disclosure to modulate 5-hydroxytryptamine (5-HT) receptors. Additionally, because the ablation of vagal efferents similarly attenuated the increase in intestinal and colonic transit observed with the compositions according to the invention, this example is consistent with the capability of compositions in accordance with the disclosure to modulate visceral afferent signaling (and our understanding that at least HT-3 receptors present on the vagus nerve are being modulated).

TABLE 3
Small intestinal and colonic transit.

| | Small intestinal, | Colonic transit, |
|---|---|---|
| Group | dye transit, cm | pellets expelled/1 h |
| C | 65.1 ± 5.2 | 3.83 ± 0.48 |
| E100 | 83.0 ± 2.7* | 5.83 ± 0.83* |
| O10E100 | 73.3 ± 5.3* | 4.33 ± 0.61 |
| VAE100 | 69.3 ± 4.4 | 4.20 ± 1.01 |

**significant at p < 0.01;
*significant at p < 0.05

### Example 6

### Demonstration of Decreased Gastric Acid Secretion

Animals were cared for and randomized into the treatment groups according to the methods described in Example 3. After an overnight fast, on day 8, the animal groups received a treatment as described in Example 1.

Thereafter, the animals were allowed free access to food, and food intake was recorded 2 h following the treatment. At this point, the animals were sacrificed and the pH of the stomach contents was recorded to estimate gastric acid production. The results are presented in Table 4 and demonstrate that the botanical extract enriched with pregnane derivatives according to the invention significantly decreased gastric acid production/secretion.

Because treatment with ondansetron (a 5-HT3 antagonist) attenuated the decrease in gastric acid production observed with the compositions according to the invention, this example is consistent with the capability of compositions in accordance with the disclosure to modulate 5-hydroxytryptamine (5-HT) receptors. Additionally, because the ablation of vagal efferents similarly attenuated the decrease in gastric acid production observed with the compositions according to the invention, this example is consistent with the capability of compositions in accordance with the disclosure to modulate visceral afferent signaling (and our understanding that at least HT-3 receptors present on the vagus nerve are being modulated).

TABLE 4
Acidity of the gastric content.

| | Gastric content pH, |
|---|---|
| Group | units |
| C | 3.62 +− 0.33 |
| E100 | 4.63 +− 0.19* |
| O10E100 | 4.03 +− 0.06 |
| VAE100 | 4.20 +− 0.25* |

**significant at p < 0.01;
*significant at p < 0.05

The invention is not limited to the embodiments described and exemplified above, but rather is capable of variation and modification without departure from the scope of the appended claims.

What is claimed is:

1. A method of treating a gastrointestinal condition, comprising:
administering a therapeutically effective amount of a composition containing an extract product of a plant material of an Asclepias plant to an individual having a gastrointestinal condition.

2. The method of claim 1, wherein the extract product comprises compounds in accordance with the following formula (I): 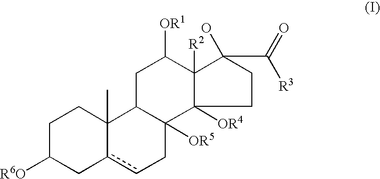
wherein R¹ is hydrogen or a C₁-C₁₈ moiety;
R² is hydrogen or a C₁-C₁₈ moiety;
R³ is a C₁-C₁₈ moiety;
R⁴ is hydrogen or a C₁-C₁₈ moiety;
R⁵ is hydrogen or a C₁-C₁₈ moiety;
R⁶ is hydrogen, a C₁-C₁₈ moiety, or a saccharide moiety; and,
the dotted line represents an optional double bond.

3. The method of claim 1, wherein the extract product comprises compounds in accordance with the following formula (II): 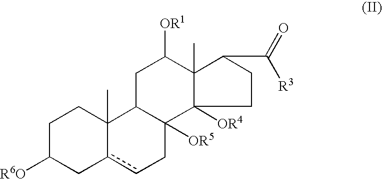
wherein R¹ is hydrogen or a C₁-C₁₈ moiety;
R³ is a C₁-C₁₈ moiety;
R⁴ is hydrogen or a C₁-C₁₈ moiety;
R⁵ is hydrogen or a C₁-C₁₈ moiety;
R⁶ is hydrogen, a C₁-C₁, moiety, or a saccharide moiety; and,
the dotted line represents an optional double bond.

4. The method of claim 1, wherein the extract product comprises compounds in accordance with the following formula (III): 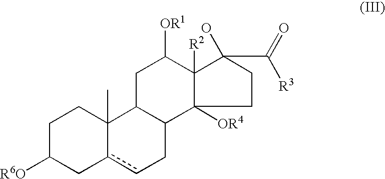
wherein R¹ is hydrogen or a C₁-C₈ moiety;
R² is hydrogen or a C₁-C₁₈ moiety;
R³ is a C₁-C₁₈ moiety;
R⁴ is hydrogen or a C₁-C₁₈ moiety;
R⁶ is hydrogen, a C₁-C₁₈ moiety, or a saccharide moiety; and,
the dotted line represents an optional double bond.

5. The method of claim 1, wherein the plant material is selected from the group consisting of leaves, stems, flowers, fruits and roots.

6. The method of claim 1, wherein the plant is selected from the group consisting of A. incarnata, A. curassayica, A. syriaca and A. tuberosa.

7. The method of claim 1, wherein the individual is a mammal.

8. The method of claim 1, wherein the individual has a gastrointestinal condition selected from the group consisting of conditions involving delayed small intestinal and/or colonic transit, conditions involving excess gastric acid production, and conditions involving rapid gastric emptying.

9. The method of claim 1, wherein the individual has a gastrointestinal condition selected from the group consisting of ileus, intestinal pseudoobstruction, Ogilivie syndrome, Hirschsprung's disease, intestinal neuronal dysplasia, gastroparesis, acid reflux disease, irritable bowel syndrome, inflammatory bowel disease, dyspepsia, emesis, and constipation.

10. The method of claim 1, wherein the composition further comprises at least one of additive selected from the group consisting of diluents, fillers, salts and binders.

11. A method of inhibiting gastric acid secretion, comprising:
administering a therapeutically effective amount of a composition containing an extract product of a plant material of an Asclepias plant to an individual in need of gastric acid secretion inhibition.

12. The method of claim 11, wherein the extract product comprises steroidal compounds.

13. The method of claim 11, wherein the plant is selected from the group consisting of A. incarnata, A. curassayica, A. syriaca and A. tuberosa.

14. The method of claim 11, wherein the individual is a mammal.

15. The method of claim 11, wherein the composition further comprises at least one of additive selected from the group consisting of diluents, fillers, salts and binders.

16. A method of treating an evacuation disorder, comprising:
administering a therapeutically effective amount of a composition containing an extract product of a plant material of an Asclepias plant to an individual having an evacuation disorder.

17. The method of claim 16, wherein the extract product comprises steroidal compounds.

18. The method of claim 16, wherein the plant is selected from the group consisting of A. incarnata, A. curassayica, A. syriaca and A. tuberosa.

19. The method of claim 16, wherein the individual is a mammal.

20. The method of claim 16, wherein the composition further comprises at least one of additive selected from the group consisting of diluents, fillers, salts and binders.
